(19) United States
(12) Patent Application Publication
(10) Pub. No.: US 2023/0000107 A1
(43) Pub. Date: Jan. 5, 2023

(54) FOODSTUFFS

(71) Applicant: MARLOW FOODS LIMITED, North Yorkshire (GB)

(72) Inventors: Muyiwa AKINTOYE, North Yorkshire (GB); Alexander James EVANS, North Yorkshire (GB); Ian DRUMMOND, North Yorkshire (GB)

(21) Appl. No.: 17/779,976

(22) PCT Filed: Nov. 27, 2020
(86) PCT No.: PCT/GB2020/053041

§ 371 Date: May 25, 2022

(30) Foreign Application Priority Data
Nov. 29, 2019 (GB) 1917488.7

(51) Int. Cl. A23J 3/20; A23J 3/22; A23J 3/26; A23L 31/00; A23J 3/14; A23P 30/25; C12N 1/14

(52) U.S. Cl. CPC A23J 3/20; A23J 3/227; A23J 3/26; A23L 31/00; A23J 3/14; A23P 30/25; C12N 1/145; A23V 2002/00; C12R 2001/77

(57) ABSTRACT

A method of making a foodstuff comprises selecting a mass comprising an edible filamentous fungus, especially of Fusarium venenatum; selecting an ingredient (A) which may be pea protein; and processing said mass and ingredient (A) in an extruder cooker to produce an extruded foodstuff.

This invention relates to a foodstuff and particularly, although not exclusively, relates to a foodstuff which is meat-like and/or may be used as a meat substitute. The invention also extends to a method of making the foodstuff and a foodstuff made in the process. Preferred embodiments include use of a filamentous fungus.

It is known, for example from WO 00/15045 (DSM), WO96/21362 (Zeneca) and WO95/23843 (Zeneca) to use edible filamentous fungi as meat-substitutes, for example in the preparation of burgers and sausages. In such uses, filaments of the fungi are bound together, for example with egg albumin, and are texturised so that the product resembles muscle fibres and therefore has a meat-like appearance and texture. Meat substitutes of the type described have been widely commercially available for many years under the trade mark QUORN.

In some circumstances, it is desirable to reduce or even eliminate the amount of egg albumin used with edible fungus in the manufacture of meat-substitutes for example on cost grounds or to produce a product suitable for vegans. It may similarly be desirable to reduce the levels of other binding agents or rheology improving agents used. GB2516491A describes edible formulations which may include reduced levels of egg albumin. To achieve a reduction, the edible formulation incorporates divalent or trivalent cations for example calcium ions. However, it is difficult to eliminate use of egg albumin completely and produce a product suitable for vegans or other individuals for whom egg-based products are unacceptable.

Another binder which may be used with filamentous fungus is agar as described in GB2551738.

Whilst there are available a wide range of foodstuffs based on filamentous fungus, for example sold under the brand name QUORN™, it is an ongoing challenge to produce foodstuffs and processed foods which closely mimic meat and/or include components which closely mimic meat, in terms of physical and/or textural properties such as hardness, resilience, cohesiveness, springiness and chewiness.

As a general point, filamentous fungus, for example, which consist essentially of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, Va.) have a cell wall comprising chitin, amongst other materials, which can make it difficult to process the fungus and/or combine it with other ingredients to achieve suitable rheological properties and/or a meat-like appearance and texture. Thus, it can be challenging to produce foodstuffs or components for foodstuffs which very closely mimic meaty textures using such filamentous fungus.

It is an object of the present invention to address the above described problems.

It is an object of the present invention to address the problem of producing foodstuffs which closely mimic meat in terms of physical and/or textural properties such as hardness, resilience, cohesiveness, springiness and/or chewiness.

It is an object of the present invention to produce foodstuffs which have improved hardness and/or chewiness/fibrosity compared to prior filamentous fungus-based foodstuffs

According to a first aspect of the invention, there is provided a method of making a foodstuff, the method comprising:
(i) selecting a mass comprising an edible filamentous fungus;
(ii) selecting an ingredient (A);
(iii) processing said mass and ingredient (A) in an extruder to produce an extruded foodstuff.

Said extruder is preferably an extruder cooker.

Said mass preferably comprises particles of said filamentous fungus (herein also referred to as “fungal particles”). Said filamentous fungus preferably comprises fungal mycelia and suitably at least 80 wt %, preferably at least 90 wt %, more preferably at least 95 wt % and, especially, at least 99 wt % of the fungal particles in said mass comprise fungal mycelia. Some filamentous fungi may include both fungal mycelia and fruiting bodies. Said fungal particles preferably comprise a filamentous fungus of a type which does not produce fruiting bodies. Where, however, a filamentous fungus of a type which produces fruiting bodies is used, the fungal particles in said mass suitably include at least 80 wt %, preferably at least 90 wt %, more preferably at least 95 wt % of fungal mycelia. Preferably, said fungal particles comprise substantially only fungal mycelia—that is, said fungal particles in said mass preferably do not include any fruiting bodies.

Preferred fungi for said fungal particles have a cell wall which includes chitin and/or chitosan. Preferred fungi have a cell wall which includes polymeric glucosamine. Preferred fungi have a cell wall which includes β1-3 and 1-6 glucans.

Said fungal particles preferably comprise (preferably consist essentially of) fungus, for example selected from fungi imperfecti.

Preferably, said fungal particles comprise, and preferably consist essentially of, cells of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, Va.) as described for example in WO96/21361 (Zeneca) and WO95/23843 (Zeneca).

Preferably, said fungal particles are non-viable. Preferably, said fungal particles have been treated to lower the level of RNA which they contain. Thus, the level of RNA in the fungal particles used is preferably less than the level in an identical fungus when in a viable state.

The level of RNA in the fungal particles is preferably less than 2 wt % on a dry matter basis.

Fungal particles in said mass may comprise filaments having lengths of less than 1000 μm, preferably less than 800 μm. Said filaments may have a length greater than 100 μm, preferably greater than 200 μm. Preferably, fewer than 5 wt %, preferably substantially no, fungal particles in said mass have lengths of greater than 5000 μm; and preferably fewer than 5 wt %, preferably substantially no, fungal particles have lengths of greater than 2500 μm. Preferably, values for the number average of the lengths of said fungal particles in said mass are also as stated above.

Fungal particles in said mass may comprise filaments having diameters of less than 20 μm, preferably less than 10 μm, more preferably 5 μm or less. Said filaments may have diameters greater than 1 μm, preferably greater than 2 μm. Preferably, values for the number average of said diameters of said fungal particles in said mass are also as stated above.

Fungal particles in said mass may comprise filaments having an aspect ratio (length/diameter) of less than 1000, preferably less than 750, more preferably less than 500, especially of 250 or less. The aspect ratio may be greater than 10, preferably greater than 40, more preferably greater than 70. Preferably, values for the average aspect ratio of said fungal particles (i.e. the average of the lengths of the particles divided by the average of the diameters of the fungal particles) in said mass are also as stated above.

Said mass may comprise said filamentous fungus and water which is suitably homogenous. The mass is preferably in the form of a paste (suitably a homogenous paste) which is suitably flowable. The viscosity of said paste at 800 Pa and 10° C. may be at least 5000, preferably at least 8000 Pa/s. The viscosity of said paste at 800 Pa and 10° C. may be less than 20000, preferably less than 13000 Pa/s. Said mass may comprise at least 10 wt % and, preferably, less than 40 wt %, of said filamentous fungus on a dry matter basis. Said mass may comprise at least 60 wt % and, preferably, less than 90 wt % of water. The ratio defined as wt % of water in said mass divided by the wt % of filamentous fungus in said mass (on a dry matter basis) may be in the range 2 to 4. Said mass may comprise 10 to 40 wt % (preferably 20 to 30 wt %) of filamentous fungus on a dry matter basis and 60 to 90 wt % (preferably 70 to 80 wt %) of water.

The sum of the wt % of said filamentous fungus and water in said mass is suitably at least 90 wt %, preferably at least 95 wt %, more preferably at least 99 wt %.

Ingredient (A) may be selected from:
(i) a puree (e.g., bean puree, sweet potato puree, pumpkin puree, applesauce, yam puree, banana puree, plantain puree, date puree, prune puree, fig puree, zucchini puree, carrot puree, coconut puree);
(ii) native or modified starches (e.g., starches from grains, starches from tuber, potato starch, sweet potato starch, corn starch, waxy corn starch, tapioca starch, tapioca, arrowroot starch, taro starch, pea starch, chickpea starch, rice starch, waxy rice starch, lentil starch, barley starch, sorghum starch, wheat starch, and physical or chemical modifications thereof [including, e.g., pre-gelatinized starch, acetylated starch, phosphate bonded starch, carboxymethylated starch, hydroxypropylated starch]);
(iii) flours derived from grains or legumes or roots (e.g., from taro, banana, jackfruit, konjac, lentil, fava, lupin bean, pea, bean, rice, wheat, barley, rye, corn, sweet rice, soy, teff, buckwheat, amaranth, chickpea, sorghum, almond, chia seed, flaxseed, potato, tapioca, potato);
(iv) protein isolates (e.g., from potato, soy, pea, lentil, chickpea, lupin, oat, canola, wheat), hydrolyzed protein isolates (e.g., hydrolyzed pea protein isolate, hydrolyzed soy protein isolate);
(v) protein concentrates (e.g. from algae, lentil, pea, soy, chickpea, rice, hemp, fava bean, pigeon pea, cowpea, vital wheat gluten);
(vi) gums (e.g., xanthan gum, guar gum, locust bean gum, gellan gum, gum arabic, vegetable gum, tara gum, tragacanth gum, konjac gum, fenugreek gum, gum karaya, gellan gum, high-acetyl gellan gum, low-acetyl gellan gum);
(vii) native or relatively folded (i.e., not fully in the native functional state but not fully denatured) proteins (e.g., fava protein, lentil protein, pea protein, ribulose-1,5-bisphosphate carboxylase/oxygenase [Rubisco], chickpea protein, mung bean protein, pigeon pea protein, lupin bean protein, soybean protein, white bean protein, black bean protein, navy bean protein, adzuki bean protein, sunflower seed protein);
(viii) polysaccharides and modified polysaccharides (e.g., methylcellulose, hydroxypropyl methylcellulose, carboxymethyl cellulose, maltodextrin, carrageenan and its salts, alginic acid and its salts, agar, agarose, agaropectin, pectin, alginate).

Said ingredient (A) may be derived from a non-animal source. Said ingredient (A) may be derived from a plant. Said ingredient (A) preferably comprises a vegetable protein.

Said ingredient (A) may be derived from pea. It may comprise a pea protein which may be derived from whole pea or from a component of pea in accordance with methods generally known in the art. The pea may be standard pea (i.e., non-genetically modified pea), commoditized pea, genetically modified pea, pea flour, pea protein concentrate, pea protein isolate or combinations thereof.

Other ingredients may be processed with said mass comprising said edible fungus and said ingredient (A) (which, especially, is pea protein) to produce said foodstuff. For example, the method may comprise selecting an ingredient (B) and suitably processing said ingredient (B) with said mass and ingredient (A) in said extruder.

Ingredient (B) may be a fibre, for example a vegetable-derived fibre. It may be pea fibre, wheat fibre or potato fibre.

The method may comprise selecting an ingredient (C) and suitably processing said ingredient (C) with said mass and ingredient (A) in said extruder. Ingredient (C) may be a starch, for example a vegetable-derived starch. It may be pea starch.

In the method, one or a plurality of vegetable proteins, for example as described for ingredient (A) may be selected and processed in the method to make said foodstuff. In some embodiments, flavourants (e.g. salt) may be selected and processed in the method to make said foodstuff.

The wt % of said mass selected in step (i) based on the total weight of ingredients processed in said extruder to produce said extruded foodstuff (the total weight being referred to as the “TWI) may be at least 45 wt %, and is suitably less than 85 wt %. Said wt % of said mass based on said TWI may be in the range 50 to 85 wt %, preferably 55 to 75 wt %, more preferably 60 to 70 wt %.

The wt % of ingredient (A) (e.g. pea protein) selected in step (ii) based on the TWI may be at least 10 wt % and is, suitably, less than 55 wt %. Said wt % of ingredient (A) based on the TWI may be in the range 15 to 50 wt %, preferably 25 to 45 wt %, more preferably 30 to 40 wt %.

The sum of the wt % of ingredient (A) and any and all other vegetable proteins introduced into the extruder based on the TWI may be at least 10 wt % and is, suitably, less than 55 wt %. Said wt % of ingredient (A) and any and all other vegetable proteins introduced into the extruder based on the TWI may be in the range 15 to 50 wt %, preferably 25 to 45 wt %, more preferably 30 to 40 wt %.

A ratio (I) defined as the wt % of said mass selected in step (i) divided by the wt % of said ingredient (A) selected in step (ii) may be at least 1, preferably at least 1.8. Said ratio (I) may be in the range 1 to 10, preferably 1 to 6, more preferably 1 to 3.

A ratio (II) defined as the wt % of said mass selected in step (i) divided by the sum of the wt % of ingredient (A) and any and all other vegetable proteins processed in said extruder to produce said foodstuff may be at least 1, preferably at least 1.8. Said ratio (II) may be in the range 1 to 10, preferably 1 to 6, more preferably 1 to 3.

The sum of the wt % of said mass selected in step (i) and the wt % of ingredient (A) selected in step (ii) based on the TWI may be at least 60 wt %, at least 75 wt % or at least 90 wt %.

The sum of the wt % of said mass selected in step (i), the wt % of ingredient (A) selected in step (ii) and any and all other vegetable proteins processed in said extruder to produce said foodstuff based on the TWI may be at least 60 wt %, at least 75 wt % or at least 90 wt %.

Preferably, the total wt % of water based on the TWI, introduced into the extruder (including water included in any ingredient, for example, said mass selected in step (i)) is at least 30 wt %, preferably at least 45 wt %. Said total wt % of water may be in the range 30 to 65 w %, for example in the range 40 to 60 wt %.

Preferably, a ratio (III) defined as the wt % of said mass of edible filamentous fungus on a dry matter basis divided by the sum of the wt % of all starches processed in said extruder on a dry matter basis is greater than 1, preferably greater than 5, more preferably greater than 10.

The wt % of said mass selected in step (i) on a dry matter basis based on the total weight of ingredients processed in said extruder to produce said extruded foodstuff (the total weight being referred to as the “TWI) may be at least 10 wt %, and is suitably less than 20 wt %. Said wt % of said mass based on said TWI may be in the range 11 to 20 wt %, preferably 12 to 17 wt %, more preferably 13 to 16 wt %.

A ratio (IV) defined as the wt % of said mass selected in step (i) on a dry matter basis divided by the wt % of said ingredient (A) selected in step (ii) may be at least 0.2, preferably at least 0.4. Said ratio (IV) may be in the range 0.2 to 2.5, preferably 0.3 to 1.5, more preferably 0.3 to 0.7.

A ratio (V) defined as the wt % of said mass selected in step (i) on a dry matter basis divided by the sum of the wt % of ingredient (A) and any and all other vegetable proteins processed in said extruder to produce said foodstuff may be at least 0.2, preferably at least 0.4. Said ratio (V) may be in the range 0.2 to 2, preferably 0.3 to 1.3, more preferably 0.3 to 0.7.

Said extruder may comprise a mixed screw profile for example, it may be a twin-screw extruder, or a single screw extruder or a planetary extruder with multiple screw configuration.

After step (ii), ingredient (A) may be introduced into the extruder, for example into a mixing zone thereof. Ingredient (A) may be introduced via a first inlet into the extruder. Said mass of edible filamentous fungus may be introduced into said extruder at a position which is downstream of the position of introduction of ingredient (A). For example, said mass may be introduced via a second inlet which is suitably downstream of said first inlet.

Preferably, in the method, said mass of edible filamentous fungus and ingredient (A) are mixed in the extruder, suitably in a mixing zone thereof.

In the method, ingredient (A) and optional ingredients (B) and/or (C) (when provided) may be introduced via the first inlet suitably concurrently and/or as a mixture.

In some embodiments, said mass of edible filamentous fungus may be introduced as a component of a mixture which includes ingredient (A). In this case, said mass and ingredient (A) may be mixed, prior to step (iii).

Said mass of edible filamentous fungus may be in the form of a viscous material, for example a paste. Said mass of edible filamentous fungus may be pumped into the extruder, suitably using a positive displacement pump, such as a progressive cavity pump.

In the extruder, said mass and said ingredient (A) may be subjected to a temperature which is at least 100° C., preferably at least 120° C., more preferably at least 130° C. The temperature to which a mixture comprising said mass and said ingredient (A) is subjected preferably does not exceed 200° C. and preferably does not exceed 180° C. More preferably, said temperature does not exceed 160° C.

In the extruder, said mass of edible filamentous fungus attains a maximum temperature of less than 180° C., preferably of less than 170° C., more preferably less than 160° C. If the mass is heated to too high a temperature, it may burn.

In the extruder, the pressure may be at least 4 bar; preferably it does not exceed 30 bar.

After subjecting said mass and other ingredients to an elevated temperature in said extruder, the mixture may pass to an elongated cooling zone which may have a length of at least 0.8 m, at least 2 m, at least 4 m or at least 6 m. In the cooling zone, means for actively reducing the temperature and the heat load of the mixture may be provided.

The method may comprise exposing the mixture to the atmosphere, downstream of the cooling zone. An extrudate comprising cooked and extruded ingredients is suitably produced. The extrudate may have a length of at least 20 cm since this allows there to be expansion by steam being lost from the extrudate.

The method may include further treating the extrudate to define said foodstuff. For example, said extrudate may be comminuted to define smaller pieces which may define chunks or pieces of meat. The method may include contacting the foodstuff with other ingredients, for example flavours.

Said method may advantageously not require freeze texturization which is required when currently producing foodstuffs comprising edible filamentous fungus as described herein.

The invention extends to a foodstuff made in a method of the first aspect.

According to a second aspect of the invention, there is provided a foodstuff comprising an edible filamentous fungus and an ingredient (A).

Said edible filamentous fungus and said ingredient (A) may be as described according to said first aspect.

Fungal particles in said foodstuff may comprise filaments having lengths which are less than in fungal particles selected in step (i) of the method.

Said foodstuff may include an ingredient (B) as described according to the first aspect.

Said foodstuff may include an ingredient (C) as described according to the first aspect.

The wt % (on a dry weight basis) of edible filamentous fungus in said foodstuff based on the total weight of ingredients in said foodstuff (the total weight being referred to as the “TWF) may be at least 10 wt %, and is suitably less than 25 wt %. Said wt % of said mass based on said TWF may be in the range 7 to 30 wt %, preferably 9 to 25 wt %, more preferably 10 to 20 wt %.

The wt % of ingredient (A) (e.g. pea protein) based on the TWF may be at least 10 wt % and is, suitably, less than 55 wt %. Said wt % of ingredient (A) based on the TWF may be in the range 15 to 50 wt %, preferably 20 to 40 wt %, more preferably 21 to 35 wt %.

The sum of the wt % of ingredient (A) and any and all other vegetable proteins in said foodstuff based on the TWF may be at least 10 wt % and is, suitably, less than 55 wt %. Said wt % of ingredient (A) and any and all other vegetable proteins in said foodstuff based on the TWF may be in the range 15 to 50 wt %, preferably 25 to 45 wt %, more preferably 25 to 36 wt %.

The wt % (on a dry weight basis) of starch in said foodstuff based on the total weight of ingredients in said foodstuff (the total weight being referred to as the “TWF”) may be at least 0.1 wt %, and is suitably less than 10 wt %. Said wt % (on a dry weight basis) of starch in said foodstuff may be in the range 0.1 to 5 wt %, preferably 0.3 to 3 wt %, more preferably 0.4 to 2 wt %.

A ratio (VI) defined as the wt % of said edible filamentous fungus on a dry matter basis divided by the wt % of said ingredient (A) may be at least 0.2, preferably at least 0.4. Said ratio (VI) may be in the range 0.2 to 2.5, preferably 0.3 to 1.5, more preferably 0.25 to 0.7.

A ratio (VII) defined as the wt % of said edible filamentous fungus on a dry matter basis divided by the sum of the wt % of ingredient (A) and any and all other vegetable proteins in said foodstuff may be at least 0.2, preferably at least 0.4. Said ratio (VII) may be in the range 0.2 to 2, preferably 0.3 to 1.3, more preferably 0.3 to 0.7.

The sum of the wt % of said edible filamentous fungus on a dry matter basis mass and the wt % of ingredient (A) based on the TWF may be at least 20 wt %, at least 30 wt % or at least 35 wt %.

The texture of the foodstuff may be analysed as described herein.

Said foodstuff may have a hardness, measured as described, of at least 2500, at least 5000, at least 10000 or at least 20000. The hardness may be less than 40000.

Said foodstuff may have a resilience, measured as described, of at least 48 or at least 50 or at least 55. Said resilience may be less than 100 or less than 80.

Said foodstuff may have a cohesiveness, measured as described, of at least 0.6. Said cohesiveness may be less than 1.0.

Said foodstuff may have a springiness, measured as described, of at least 90 or at least 200. Said springiness may be less than 800.

Said foodstuff may have a chewiness of at least 5000 or at least 7000 or at least 15000. The chewiness may be less than 30000.

Said foodstuff is preferably meat-like.

According to a third aspect of the invention, there is provided apparatus for undertaking the method of the first aspect and/or for producing the foodstuff of the second aspect, the apparatus comprising:
(a) an extruder;
(b) a receptacle (I) containing a mass comprising an edible filamentous fungus, wherein said receptacle (I) is operatively connected to the extruder for transferring said mass from the receptacle (I) to the extruder;
(c) a receptacle (II) containing an ingredient (A), wherein said receptacle (II) is operatively connected to the extruder for transferring ingredient (A) from the receptacle (II) to the extruder.

Said extruder is preferably an extruder cooker.

Any feature of any aspect of any invention described herein may be combined with any feature of any other invention described herein mutatis mutandis.

Specific embodiments of the invention will now be described, by way of example, with reference to the accompanying drawings, in which:

FIG. 1 is a schematic diagram of a high moisture extrusion cooking apparatus;

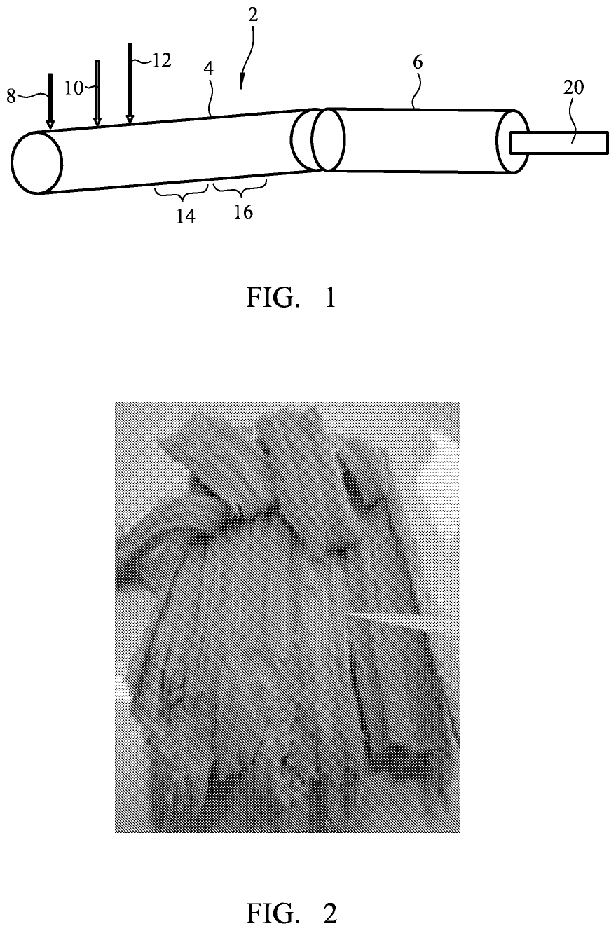

FIG. 2 is a photo of an extruded product produced without any steam expansion;

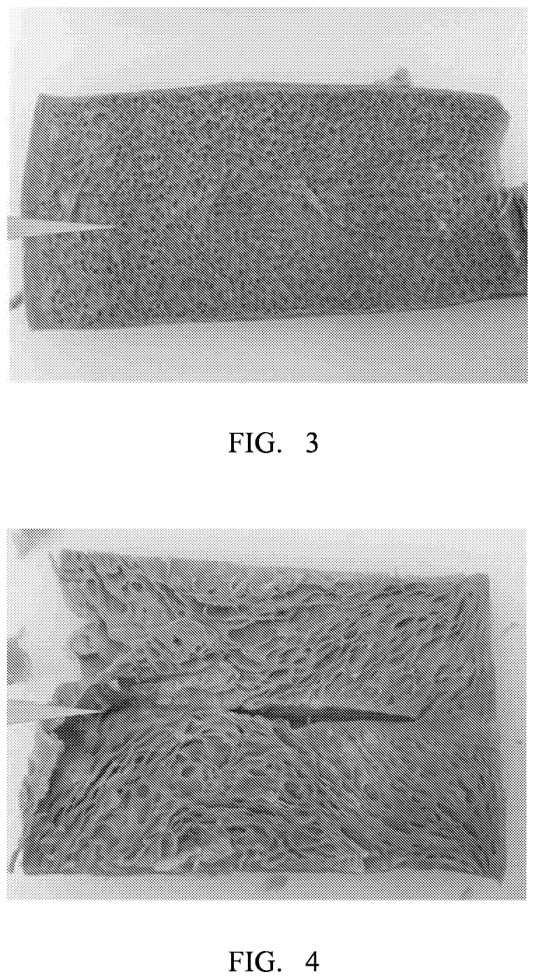

FIG. 3 is a photo of an extruded product with a level steam expansion; and

FIG. 4 is a photo of an extruded product with an enhanced level of steam expansion compared to that in FIG. 3.

The following material is referred to hereinafter:

Mycoprotein paste —Mycoprotein paste-refers to a visco-elastic material comprising a mass of edible filamentous fungus derived from Fusarium venenatum A3/5 (formerly classified as Fusarium graminearum Schwabe) (IMI 145425; ATCC PTA-2684 deposited with the American type Culture Collection, 12301 Parklawn Drive, Rockville Md. 20852) and treated to reduce its RNA content to less than 2% by weight by heat treatment. Further details on the material are provided in WO96/21362 and WO95/23843. The material may be obtained from Marlow Foods Limited of Stokesley, U.K. It comprises about 23-25 wt % solids (the balance being water) made up of non-viable RNA reduced fungal hyphae of approximately 400-750 μm length, 3-5 μm in diameter and a branching frequency of 2-3 tips per hyphal length. The paste has a viscosity, measured as described below, at 800 Pa and 10° C. of 10,462 Pa/s.

### Measurement of Viscosity

| | |
|---|---|
| Rheometer | (Malvern) Kinexus Lab+ |
| Apparatus/Geometry | 20 mm Parallel Plates (Serrated) |
| Plate Gap | 2 mm |
| Test Method | Shear Stress Ramp (rSpace V003-1) |
| Range | Shear Stress 200-1400 (Pa) |
| Temperature | 10° C. |
| Sample | Fresh Mycoprotein (23%) |

In the measurement method, a mycoprotein paste sample was placed in the rheometer and sandwiched, with a 2 mm gap, between an upper 20 mm diameter serrated parallel plate and lower flat serrated Peltier plate and cooled to the required measurement temperature. The instrument was operated in Shear Stress Ramp mode where a series of individual stresses was applied to the sample for 60 seconds and a response measured. Stress is defined as force per unit area.

Referring to FIG. 1, a high moisture extrusion cooking (HMEC) apparatus 2 comprises a twin-screw extruder 4 and, downstream thereof, a cooling and fibre alignment barrel 6. Ingredients are introduced into the extruder 4 via inlets, 8, 10, 12 and mixed by co-rotating screws of the extruder and conveyed through a series of heated zones of the extruder. By way of example, in a first heating zone 14, the temperature may be in the range 140° C. to 160° C. Downstream thereof in a second heating zone 16, the temperature may be in the range 110° C. to 130° C. Downstream of the second heating zone 16 is a pre-cooling zone.

Downstream of the pre-cooling zone, the extruder 4 is arranged to deliver a mixture into the barrel 6. Barrel 6 includes cooling channels (not shown) in which water at, for example, a temperature in the range 60° C.-85° C. may flow so that a mixture passing through the barrel 6 is slowly cooled. Extrudate 20 exiting the extruder may be at a temperature in the range 105° C.-121° C. The temperature, flow rates and/or pressures within the extruder cooker may be selected to ensure the mixture flows (and does not block) the extruder. In addition, the temperature should not be too high, thereby to avoid burning of any of the ingredients.

The length of the barrel 6 may be in the range 800 cm-3200 cm to allow extrudate to be slowly cooled during its passage through the barrel downstream of the extruder.

A typical recipe for processing in the apparatus described may be as follows:

TABLE 1

| Ingredient | wt % |
|---|---|
| Pea protein | 28.5-30.5 dry weight basis |
| isolate | |
| Pea fibre | 3.0-3.5 dry weight basis |
| Mycoprotein | 61.0-65.0 wet weight basis |
| Pea starch | 0.9-1.1 dry weight basis |
| Additional water | 0.0-0.7 |

Using the apparatus of FIG. 1, pea protein isolate and pea fibre may be introduced into the extruder. A dry mix of the ingredients may be pre-blended in a ribbon or paddle blender and then charged to a hopper of a loss in weight feeder from which the ingredients may be fed into the extruder via inlet 8. Downstream thereof, any additional water may be introduced via inlet 10 at a controlled rate. Downstream of inlet 10, mycoprotein may be introduced via inlet 12, using a high pressure positive displacement pump. The ingredients contact one another in the extruder and are mixed under conditions of high temperature, high shear and high pressure.

During passage through the extruder, the globular pea protein melts. Surprisingly, it is found that, despite the presence of its tough chitin cell wall, the mycoprotein is also sufficiently softened so that it can be homogenously mixed with and/or fragmented and/or mixed into the other ingredients.

Downstream of the extruder 4, in the cooling and fibre alignment barrel 6, the mixture is slowly cooled. During cooling, the mixture, in particular the proteins therein, appear to reassemble and eventually become set into a 3D fibrated structure that is found to deliver a meaty texture. The structure is believed to be held together by a combination of covalent, electrostatic and hydrogen bonds as well as hydrophobic interactions. The extrudate 20 which emerges from the barrel 6 is in the form of a long continuous belt having a typical moisture content in the range 45-55 wt %.

Depending on conditions used, for example the rate of cooling in the barrel 6, and how quickly steam leaves the product on exiting barrel 6, products having different appearances/properties may be produced as illustrated in FIGS. 2 to 4 and in the subsequent specific examples.

After cooling to ambient temperature, the extrudate may be size reduced by shredding, slicing, dicing, cutting or flaking and/or such comminuted foodstuff may be used as an ingredient in other products.

Table 2 summarises the conditions which may be used in two different apparatus 2 which are as described in FIG. 1.

| Machine Reference | A | B |
|---|---|---|
| Number of Temperature zones | 10 | 10 |
| Cooking zone 1 temperature range | 140° C.-160° C. | 130° C.- 45° C. |
| Cooking zone 2 temperature range | 130° C.-110° C. | 130° C.- 45° C. |
| Pre cooling zone | 125° C.-100° C. | 130° C.- 20° C. |
| Temp of material exiting extruder | 105° C.-121° C. | 129° C.- 35° C. |
| Extruder barrel pressure | 4-7 bar | 17-26 bar |
| Cooling die recirculation temperature | 85° C.-60° C. | 90° C.- 60° C. |
| Cooling die length | 800 cm | 800-3200 cm |
| Extruder screw speed | Typically | Typically |
| | 500 rpm. | 800 rpm. |
| Rotation | Co-rotating | Co-rotating |
| Throughput of mycoprotein | 2-15 kgph | 57-95 kgph |
| Throughput of pea protein & pea fibre | 2-7.5 kgph | 30-51 kgph |
| Throughput of water | 0-10 kgph | 0-6 kgph |
| Total throughput (exit die) | 11-20 kgph | 87-146 kgph |

Table 2

The following examples further illustrate the invention.

### EXAMPLES 1 TO 6

The apparatus described above was used to produce a range of different samples using the following ingredients.

| | | | Calculated barrel |
|---|---|---|---|
| Ingredient | State | wt % | water |
| Pea protein isolate | Dry | 30.5 | 1.522 |
| Pea fibre | Dry | 3.5 | 0.175 |
| Mycoprotein | Wet | 65.0 | 48.75 |
| Pea starch | Dry | 1.1 | 0.0525 |
| Water | — | 0.0 | — |

Although pea protein isolate, pea fibre and pea starch are nominally dry, they do include some water, the amount of which has been calculated and included in the table above.

The table below details the conditions used in the apparatus and provide remarks on the nature of the product.

| | | | | Temper- | | |
|---|---|---|---|---|---|---|
| | | Through- | | ature of | | |
| | Throughput | put pea | Through- | mass outlet | Motor | Amount |
| Example | Mycoprotein | material | put water | extruder | speed | cooling |
| number | (kg/h) | [kg/h] | [kg/h] | [° C.] | [1/min] | dies |
| 1 | 57 | 30 | 0 | 119 | 1300 | 4 |
| 2 | 74 | 39 | 6 | 129 | 800 | 3 |
| 3 | 57 | 30 | 2 | 123 | 1500 | 3 |
| 4 | 74 | 39 | 0 | 133 | 800 | 3 |
| 5 | 74 | 39 | 0 | 133 | 800 | 3 |
| 6 | 94.9 | 51.1 | 0 | 128 | 800 | 2 |
| | | | | | Ratio | |
| | | temperature | | Total | mycoprotein | |
| Example | Torque | cooling units | Pressure | through- | to total | |
| number | [%] | [° C.] | bar | put | throughput | Remarks |
| 1 | 18 | 70-70/60/60 | 34 | 87 | 65.5% | Product slightly expanded |
| | | | | | | (structure is not constant) |
| 2 | 23 | 80-80-70- | 26 | 119 | 62.2% | Good expansion over entire |
| | | | | | | product |
| 3 | 19 | 70-70-60- | 17 | 89 | 64.0% | Comparable to Example 2, |
| | | | | | | good flow, very small |
| | | | | | | expansion |
| 4 | 22 | 80-80-70- | 22 | 113 | 65.5% | Big bubbles in the middle, |
| | | | | | | outside hard |
| 5 | 22 | 80-80-90- | 22 | 113 | 65.0% | Good expansion, also |
| | | | | | | product edge is slightly |
| | | | | | | expanded; product is not so |
| | | | | | | flaky |
| 6 | 26 | 60-61 | 23 | 146 | 65.0% | Product is more torn and |
| | | | | | | more irregular flow with the |
| | | | | | | additon of starch |

Products produced were tested as described in Example 7.

### EXAMPLE 7—TEXTURE PROFILE ANALYSIS (TPA)

Products produced as described in Example 1 to 6 were cut into 25 mm×25 mm squares to define samples for testing. The samples were of varying thickness, ranging from 10 mm-20 mm, dependent on the extrusion method that had been used to produce the products. All samples were defrosted from a frozen state in a 4° C. chiller for 12 hours prior to analysis and were analysed within 10 minutes of removal from chill hold.

TPA was performed using a TA. XT Plus Texture Analyser (Stable Micro Systems, Godalming UK) and a stainless-steel compression platen of 75 mm diameter (Stable Micro Systems, Godalming UK). The platen attachment was used to compress each sample using the standard ‘Simplified TPA’ method, found within the Exponent software from Stable Micro Systems; a modified version of the original instrumental test method created by A. Szczesniak (1963) and General Foods Corporation Technical Centre in 1963- see SZCZESNIAK, A. S., BRANDT, M. A. and FRIEDMAN, H. H. (1963), Development of Standard Rating Scales for Mechanical Parameters of Texture and Correlation Between the Objective and the Sensory Methods of Texture Evaluation. Journal of Food Science, 28: 397-403.

Parameters used for the method were as detailed in the table below.

| | |
|---|---|
| Pre-Test Speed | 5.00 mm/sec |
| Test Speed | 3.00 mm/sec |
| Post-Test Speed | 5.00 mm/sec |
| Compression Percentage | 35% |
| Time Between Compressions | 5.00 seconds |
| Trigger Force | 20 g |

Samples were compressed using the compression platen to a percentage of 35% at a speed of 3.00 mm/sec, using a 2-cycle analysis which allowed a 5.00 second gap between compressions. Extrusion samples were benchmarked to current commercial Quorn™ Vegetarian and Vegan Pieces. The deformation curve of each sample was obtained, and results used to determine the mechanical parameters of the samples, including; Hardness, Resilience, Cohesiveness, Springiness and Chewiness. The five characteristics were calculated by the ‘Simplified TPA’ macro included in the Exponent software from Stable Micro Systems. Each textural/mechanical parameter is explained below in Table 2, with reference to Texturetechnologies.com. (2019). Texture Profile Analysis. [online] Available at: https://texturetechnologies.com/resources/texture-profile-analysis#select-characteristics [Accessed 5 Nov. 2019].

| | |
|---|---|
| Hardness | Peak Force 1 |
| Resilience | Area 4/Area 3 |
| Cohesiveness | Area 2/Area 1 |
| Springiness | Distance 2/Distance 1 |
| Chewiness | Hardness × Cohesiveness × Springiness |

### Results

TPA Results for Examples 1 to 6 and for the two commercial Quorn™ control samples are provided in the table below. The table shows the five texture characteristics measured using the TPA method.

| Example No. | Hardness (N) | Resilience | Cohesiveness | Springiness | Chewiness (N) |
|---|---|---|---|---|---|
| 1 | 29437 | 61 | 0.86 | 200 | 53900 |
| 2 | 9226 | 54 | 0.85 | 119 | 8207 |
| 3 | 31047 | 59 | 0.86 | 93 | 24931 |
| 4 | 24154 | 58 | 0.69 | 79 | 10515 |
| 5 | 29410 | 51 | 0.80 | 93 | 21811 |
| 6 | 1353 | 52 | 0.93 | 664 | 8325 |
| QUORN ™ | 2442 | 45 | 0.84 | 97 | 1976 |
| Vegetarian | | | | | |
| Standard | | | | | |
| QUORN ™ | 1691 | 38 | 0.76 | 93 | 1187 |
| Vegan Standard | | | | | |

The results show that the apparatus can produce products with advantageous properties which may surpass the properties of current commercially-available QUORN™ products. In addition, these additional properties provide the option of tailor making the texture to suit other downstream process such as shredded or pulled meat. This is not possible using current techniques used for making commercially-available QUORN™ products. FIGS. 2 to 4 illustrate different textures that may be obtained. Example 5 may, in some circumstances, be a preferred product due to its combination of properties. The sample advantageously has higher hardness comparable resilience, cohesiveness and springiness and higher chewiness compared to the commercial QUORN™ products.

Products produced as described may be further processed into commercial products such as mince, chunks or shredded pieces by addition of other ingredients such as flavourants, fats, oils, marinades, coatings etc.

Using machines A and B as described allows up to 70 wt % of mycoprotein to be incorporated into the mixture to produce an even, homogenous, fibrous mass of product.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of making a foodstuff, the method comprising:
(i) selecting a mass comprising an edible filamentous fungus;
(ii) selecting an ingredient (A);
(iii) processing said mass and ingredient (A) in an extruder to produce an extruded foodstuff.

2. A method according to claim 1, wherein said extruder is an extruder cooker, wherein, in the method, said mass of edible filamentous fungus and ingredient (A) are mixed in the extruder.

3. A method according to claim 1, wherein said mass comprises particles of said filamentous fungus (herein also referred to as “fungal particles”) and said fungal particles comprise cells of Fusarium species, optionally of Fusarium venenatum A3/5.

4. (canceled)

5. A method according to claim 1, wherein said mass comprises 10 to 40% of filamentous fungus, 60 to 90% water; and/or has a viscosity at 800 Pa and 10° C. of at least 5000 and less than 20000,

6. A method according to claim 1, wherein ingredient (A) is selected from:
a puree;
a starch;
a flour;
a protein concentrate;
a protein isolate;
a gum;
a native or relatively folded protein;
a polysaccharide.

7. A method according to claim 1, wherein said ingredient (A) is derived from a non-animal source and/or wherein said ingredient (A) is derived from a plant and/or comprises a vegetable protein, optionally derived from pea.

8. (canceled)

9. A method according to claim 1, wherein said method comprises selecting an ingredient (B) and processing said ingredient (B) with said mass and ingredient (A) in said extruder, wherein ingredient (B) is a fibre, optionally a vegetable-derived fibre.

10. (canceled)

11. A method according to claim 1, wherein said method comprises selecting an ingredient (C) and processing said ingredient (C) with said mass and ingredient (A) in said extruder, wherein ingredient (C) is a vegetable-derived starch.

12. (canceled)

13. A method according to claim 1, wherein:
the wt % of said mass selected in step (i) based on the total weight of ingredients processed in said extruder to produce said extruded foodstuff (the total weight being referred to as the “TWI”) is at least 20 wt % and is less than 85 wt %; and/or
the wt % of ingredient (A) selected in step (ii) based on the TWI is at least 10 wt % and is less than 55 wt %; and/or
the sum of the wt % of ingredient (A) and any and all other vegetable proteins introduced into the extruder based on the TWI is at least 10 wt % and is less than 55 wt %.

14. A method according to claim 13, wherein: the sum of the wt % of said mass selected in step (i) and the wt % of ingredient (A) selected in step (ii) based on the TWI is at least 60 wt %; and/or wherein the sum of the wt % of said mass selected in step (i), the wt % of ingredient (A) selected in step (ii) and any and all other vegetable proteins processed in said extruder to produce said foodstuff based on the TWI is at least 60 wt %.

15. A method according to claim 1, wherein a ratio (I) defined as the wt % of said mass selected in step (i) divided by the wt % of said ingredient (A) selected in step (ii) is at least 1; and/or
a ratio (II) defined as the wt % of said mass selected in step (i) divided by the sum of the wt % of ingredient (A) and any and all other vegetable proteins processed in said extruder to produce said foodstuff is at least 1.

16. A method according to claim 13, wherein the total wt % of water based on the TWI, introduced into the extruder is at least 30 wt %.

17. A method according to claim 1, wherein a ratio (Ill) defined as the wt % of said mass of edible filamentous fungus on a dry matter basis divided by the sum of the wt % of all starches processed in said extruder on a dry matter basis is greater than 1.

18. A method according to claim 1, wherein a ratio (IV) defined as the wt % of said mass selected in step (i) on a dry matter basis divided by the wt % of said ingredient (A) selected in step (ii) is at least 0.2.

19. A method according to claim 1, wherein, in the extruder, said mass of edible filamentous fungus attains a maximum temperature of less than 180° C.

20. A method according to claim 1, wherein a ratio (V) defined as the wt % of said mass selected in step (i) on a dry matter basis divided by the sum of the wt % of ingredient (A) and any and all other vegetable proteins processed in said extruder to produce said foodstuff is in the range 0.2 to 2.

21. A method according to claim 1, wherein after subjecting said mass and other ingredients to an elevated temperature in said extruder, the mixture passes to an elongated cooling zone which has a length of at least 0.8 m; and/or the method comprises comminuting said extrudate to define smaller pieces; and/or the method includes contacting the foodstuff with other ingredients.

22. A meat-like foodstuff which is an extrudate comprising an edible filamentous fungus and an ingredient (A), wherein ingredient (A) is selected from:
a puree;
a starch;
a flour;
a protein concentrate;
a protein isolate;
a gum;
a native or relatively folded protein;
a polysaccharide;
wherein said ingredient (A) is derived from a non-animal source;
wherein said foodstuff has a hardness of at least 2500; and
said filamentous fungus comprises cells of Fusarium species.

23. (canceled)

24. (canceled)

25. (canceled)

26. (canceled)

27. (canceled)

28. (canceled)

29. (canceled)

30. A method according to claim 1, wherein:
a ratio (IV) defined as the wt % of said mass selected in step (i) on a dry matter basis divided by the wt % of said ingredient (A) selected in step (ii) is at least 0.2;
in the extruder, said mass of edible filamentous fungus attains a maximum temperature of less than 180° C.;
a ratio (V) defined as the wt % of said mass selected in step (i) on a dry matter basis divided by the sum of the wt % of ingredient (A) and any and all other vegetable proteins processed in said extruder to produce said foodstuff is at least 0.2;
after subjecting said mass and other ingredients to an elevated temperature in said extruder, the mixture passes to an elongated cooling zone which has a length of at least 0.8 m.

31. A method of making a foodstuff, the method comprising:
(i) selecting a mass comprising an edible filamentous fungus;
(ii) selecting an ingredient (A);
(iii) processing said mass and ingredient (A) in an extruder to produce an extruded foodstuff;
wherein:
said extruder is an extruder cooker;
said mass comprises particles of said filamentous fungus which comprise cells of Fusarium species;
said mass comprises 10 to 40% of filamentous fungus and 60 to 90% water;
ingredient (A) is selected from: a puree, a starch, a flour, a protein concentrate, a protein isolate, a gum, a native or relatively folded protein, a polysaccharide;
said ingredient (A) is derived from a non-animal source; and
said method comprises selecting an ingredient (B) and processing said ingredient (B) with said mass and ingredient (A) in said extruder, wherein ingredient (B) is a fibre.
